(19) United States
(12) Patent Application Publication
(10) Pub. No.: US 2013/0053284 A1
(43) Pub. Date: Feb. 28, 2013

(54) Modular Roller Oven and Associated Methods

(71) Applicants: Dale E. Jamison, Humble, TX (US); Kenneth Heidt Matthews, Kingwood, TX (US); Ketan Chimanlal Bhaidasna, Houston, TX (US)

(72) Inventors: Dale E. Jamison, Humble, TX (US); Kenneth Heidt Matthews, Kingwood, TX (US); Ketan Chimanlal Bhaidasna, Houston, TX (US)

(21) Appl. No.: 13/222,374

(22) Filed: Aug. 31, 2011

(51) Int. Cl. E21B 47/10; C09K 8/42; G01N 33/38; C09K 8/00

(52) U.S. Cl. 507/200; 106/638; 73/152.18; 73/866

(57) ABSTRACT

A housing may include a test cell being enclosed and having at least one test cell wall and one sealable opening and has a cell central axis defined along the center line of the length of the test cell, a thermal element in thermal communication with the test cell, and an insulation, at least a portion of the insulation being disposed about the test cell and the thermal element. An apparatus may generally include a housing, a drive connection, and a shaft operably connected to the test cell and extending parallel to the cell central axis from the test cell through the insulation to the drive connection to which the shaft is operably connected. Testing a sample in the test cell of an apparatus operably connected to a driving mechanism may generally include at least manipulating the test cell and analyzing the sample.

### BACKGROUND

The present invention relates to configurations of a modular roller oven and methods relating thereto.

Roller ovens are used for, inter alia, simulating dynamic-aging of fluids. Dynamic-aging is a test in which a fluid sample is mildly agitated by rolling (or tumbling) for the duration of the test, usually performed at a selected high temperature. Typically, the fluid sample is sealed in a fluid-aging cell, often under pressure, and placed in an oven that will roll (or tumble) the fluid-aging cell continually for a given period of time, e.g., often 16 hours or overnight. The properties of the aged fluid sample are then measured. Dynamic-aging testing is especially important for fluids to be used in oilfield applications as the test simulates circulation of a fluid in a wellbore during pumping, which necessarily involves heat and pressure.

Generally, traditional roller ovens are large ovens with a series of rollers inside that can continuously roll a fluid-aging cell 360°. The volume of the roller oven may be large enough to accommodate four to six fluid-aging cells in a single horizontal plane, e.g., greater than 2 feet in width, depth, and height. Referring to FIG. 1, a nonlimiting example of a traditional roller oven, traditional roller oven 100 may generally be box 120 with rollers 130 parallel to box ceiling 124 that operably rotate about an axis perpendicular to box back wall 126, heating elements 140 located at box sidewalls 122, and fan 150 located at box ceiling 124. Further, traditional roller oven 100 may also have a control box 160 with temperature controller 162 operably connected to heating elements 140 in order to maintain box 120 at a desired temperature. Dynamic-aging tests may include placing fluid-aging cell 110 on rollers 130, rotating rollers 130, heating box 120 to a desired temperature, and maintaining said temperature for a desired length of time after which box 120 is cooled for fluid-aging cell 110 retrieval. The heat distribution system of traditional roller oven 100 with fan 150 at box ceiling 124 and heating elements 140 at box sidewalls 122 provides uneven heating to individual fluid-aging cells 110, i.e., fluid-aging cells 110 closest to heating elements 140 may be at a different temperature than fluid-aging cells 110 under fan 150. Further, a thermocouple (not shown) for monitoring the temperature in box 120 is often not near fluid-aging cells 110, so the temperature of box 120 may not be the temperature of fluid-aging cells 110. The thermal inconsistence between fluid-aging cells 110 and within box 120 are magnified at high temperatures, e.g., above about 400° F. Further, given the size of traditional roller oven 100 configured to hold four to six fluid-aging cells, heating to high temperatures, e.g., above about 400° F., takes a long time and is energy-intensive. Operating at the elevated temperatures is a further safety concern for workers using or in the vicinity of a large 400° F. oven.

This configuration also allows for only a single set of parameters to be tested at a given time. Given the plurality of subterranean formations and methods of drilling, often a variety of variables need to be tested, e.g., temperature, time, and movement. In order to test several variables with a traditional roller oven, several experiments would need to be run consecutively or with several ovens, which can be time-consuming and/or costly. Further, traditional roller ovens only provide for investigating condition variables of temperature, fluid-aging cell internal pressure, and rolling speed. In situ monitoring capabilities are not available to help understand how a fluid is aging. Only beginning- and ending-points are available to a researcher. To fill-in the gaps, additional tests are required.

Given the plurality of variables that may need to be investigated to simulate the wide array of subterranean formations, a modular system for dynamic-aging of samples where the conditions of a single cell can be manipulated, even programmed and/or monitored, would be of value to one skilled in the art.

### SUMMARY OF THE INVENTION

The present invention relates to configurations of a modular roller oven and methods relating thereto.

In some embodiments of the present invention, an apparatus may comprise: a housing that comprises a test cell being enclosed and having at least one test cell wall and one sealable opening and has a cell central axis defined along the center line of the length of the test cell, a thermal element in thermal communication with the test cell, and an insulation, at least a portion of the insulation being disposed about the test cell and the thermal element; a drive connection; and a shaft operably connected to the test cell and extending parallel to the cell central axis from the test cell through the insulation to the drive connection to which the shaft is operably connected.

In some embodiments of the present invention, a modular system may comprise: at least one apparatus that comprises a housing that comprises a test cell being enclosed and having at least one test cell wall and one sealable opening and has a cell central axis defined along the center line of the length of the test cell, a thermal element in thermal communication with the test cell, and an insulation, at least a portion of the insulation being disposed about the test cell and the thermal element; a drive connection; and a shaft operably connected to the test cell and extending parallel to the cell central axis from the test cell through the insulation to the drive connection to which the shaft is operably connected; and a frame that houses at least a portion of the housing; and a driving mechanism operably connected to the drive connection.

In some embodiments of the present invention, a method may comprise: providing an apparatus that comprises a housing that comprises a test cell being enclosed and having at least one test cell wall and one sealable opening and has a cell central axis defined along the center line of the length of the test cell, a thermal element in thermal communication with the test cell, and an insulation, at least a portion of the insulation being disposed about the test cell and the thermal element; a drive connection; and a shaft operably connected to the test cell and extending parallel to the cell central axis from the test cell through the insulation to the drive connection to which the shaft is operably connected; providing a driving mechanism operably connected to the drive connection; providing a sample in the test cell of the apparatus; manipulating the test cell; and analyzing the sample.

In some embodiments of the present invention, a method of testing the stability of deep-sea treatment fluids may comprise: providing an apparatus that comprises a housing that comprises a test cell being enclosed and having at least one test cell wall and one sealable opening and has a cell central axis defined along the center line of the length of the test cell, a thermal element in thermal communication with the test cell, and an insulation, at least a portion of the insulation being disposed about the test cell and the thermal element; a drive connection; and a shaft operably connected to the test cell and extending parallel to the cell central axis from the test cell through the insulation to the drive connection to which the shaft is operably connected; providing a driving mechanism operably connected to the drive connection; providing a sample in the test cell of the apparatus; manipulating the test cell; changing the sample temperature to a first temperature below room temperature; changing the sample temperature to a second temperature above room temperature; and analyzing the sample.

In some embodiments of the present invention, a method of testing a cement composition may comprise: providing an apparatus that comprises a housing that comprises a test cell being enclosed and having at least one test cell wall and one sealable opening and has a cell central axis defined along the center line of the length of the test cell, a thermal element in thermal communication with the test cell, and an insulation, at least a portion of the insulation being disposed about the test cell and the thermal element; a drive connection; and a shaft operably connected to the test cell and extending parallel to the cell central axis from the test cell through the insulation to the drive connection to which the shaft is operably connected; providing a driving mechanism operably connected to the drive connection; providing a cementitious sample in the test cell of the apparatus; manipulating the test cell; changing the sample temperature; and monitoring the temperature within the test cell.

The features and advantages of the present invention will be readily apparent to those skilled in the art upon a reading of the description of the preferred embodiments that follows.

### BRIEF DESCRIPTION OF THE DRAWINGS

The following figures are included to illustrate certain aspects of the present invention, and should not be viewed as exclusive embodiments. The subject matter disclosed is capable of considerable modification, alteration, and equivalents in form and function, as will occur to those skilled in the art and having the benefit of this disclosure.

FIG. 1 illustrates an example of a traditional roller oven.

FIG. 2 illustrates a nonlimiting example of a cross-sectional view of a modular roller oven of the present invention.

FIG. 3 illustrates a nonlimiting example of a cross-sectional view of a modular roller oven of the present invention.

FIG. 4 illustrates a nonlimiting example of a side view of a modular roller oven of the present invention with a view window through the base.

FIG. 5 illustrates a nonlimiting example of a side view of a modular roller oven of the present invention with extendable legs.

FIG. 6 illustrates a nonlimiting example of a single modular roller oven of the present invention.

FIG. 7 illustrates a nonlimiting example of a system with two modular roller ovens of the present invention.

FIG. 8A illustrates a nonlimiting example of a system with four modular roller ovens of the present invention.

FIG. 8B illustrates a nonlimiting example of a front view of a system with four modular roller ovens of the present invention.

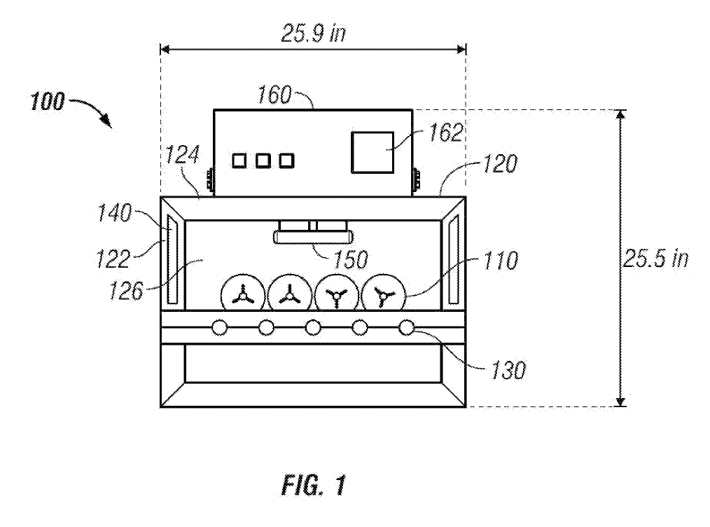

### DETAILED DESCRIPTION

The present invention relates to configurations of a modular roller oven and methods relating thereto.

In some embodiments, the present invention provides a modular roller oven as an apparatus and as part of a system. The configurations the modular roller oven provided herein function for similar purposes of traditional roller ovens. However, because of the design and size that minimizes the volume of air to heat, the modular roller oven advantageously can ramp to operating temperatures faster, can cool to manageable temperatures faster, and requires less energy to do so. Additionally, the motor used to manipulate the smaller modular roller oven may be smaller and consequently quieter.

Further, the unique integration of elements and components allows for increased flexibility and capabilities over traditional roller ovens, e.g., monitoring and manipulating samples in situ. Such elements and component described herein includes sensor that provide real-time analysis of the sample during a test. Further, traditional roller elements have been integrated in ways to increase the available capabilities including, but not limited to, variable movement of the fluid-aging cell (both speed and type), angular rolling, remote monitoring and/or manipulation, and data logging.

The modular nature of the roller oven allows for systems with more than one modular roller oven that can be operated (independently or cooperatively) at different conditions. To achieve this with traditional roller ovens would be an expensive proposition to acquire another roller oven, not to mention the floor space required. The modular roller oven configurations also provide for arranging the modular roller ovens in various configurations that can be adapted to a site (laboratory or in the field) based on, inter alia, available space and proximity to power. Further, the modular roller ovens may be designed to operate with a test cell of desired size. By way of nonlimiting example, a test cell may be a cylinder 3 inches in diameter and 10 inches in length. By way of nonlimiting example, a test cell may be a cylinder configured to test about 350 mL of sample. This size may be scalable to a desired size, and configuration as described below, with an appropriately scale modular roller oven.

The modular nature of the roller oven also allows for enhanced reliability. Operational reliability may manifest in repeatable, stable temperature of the sample in test cell. Other operation reliability may be realized if a module should become inoperable or require maintenance, only one roller oven of the system will be effected.

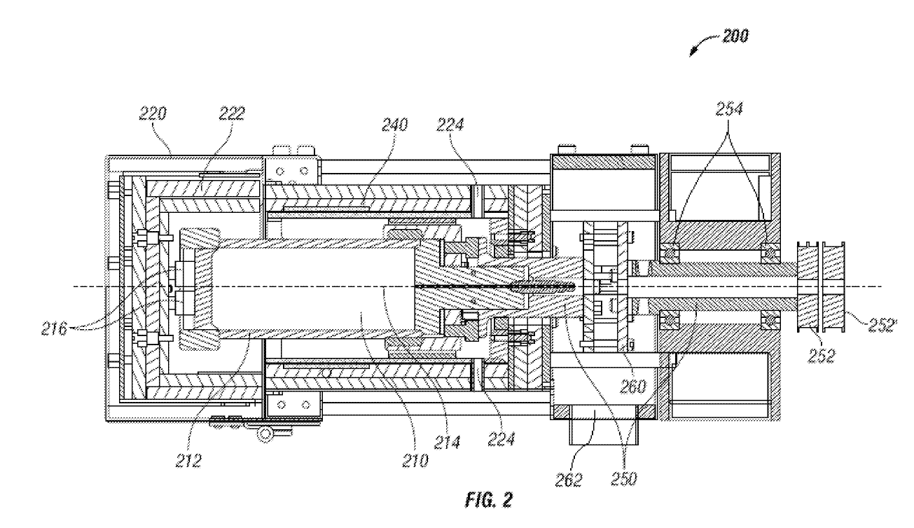

Referring now to FIG. 2, a nonlimiting embodiment of the present invention, apparatus 200 is designed to overcome many of the drawbacks and/or limitations of traditional roller oven 100. Apparatus 200 may comprise housing 220 that contains insulation 222, test cell 210, and thermal element 240 in thermal communication with test cell 210. At least a portion of insulation 222 may be disposed between housing 220 and thermal element 240. At least a portion of insulation 222 may be disposed between housing 220 and test cell 210. Test cell 210 may be enclosed with at least one cell wall 212 and may have cell central axis 214 defined along the centerline of the length of test cell 210. It should be noted that defining the centerline along the length of test cell 210 does not preclude test cells with other configurations where length may be ambiguous, e.g., spherical. One skilled in the art, with the benefit of this disclosure, should understand how to define a cell central axis for other test cell configurations.

Referring again to FIG. 2, in some embodiments, housing 220 may further comprise at least one housing port 224 that extends from outside housing 220 to inside housing 220. In some embodiments, housing port 224 may extend from outside housing 220 to inside housing 220 near test cell 210. Housing port 224 may be used for several purposes including, but not limited to, a path for an air flow to assist in cooling after a test and/or a path for a sensor (described further below).

Housing 220 may be made of any known material that is compatible with the operational requirements of apparatus 200 including temperature and movement (discussed below). Suitable materials for housing 220 include, but are not limited to, metals and metal alloys like aluminum and stainless steel; plastics and polymers like polyether ether ketone (PEEK) and polyurethane; composites including those with additives like carbon fibers, glass fibers, and nanomaterials; fiberglass; and any combination thereof. In some embodiments, housing 220 may be coated. Further, housing 220 may have a layered structure where at least some of the layers provide both structural support and insulation for apparatus 200. One skilled in the art with the benefit of this disclosure should understand the plurality of materials and configurations with which housing 220 may be designed.

Insulation 222 may be made of any known material that can serve to maintain the desired temperature of elements encompassed by insulation 222 and to minimize thermal communication between thermal element 240 and the environment external to apparatus 200. Suitable materials for insulation 222 may include, but not be limited to, glass and fiberglass; graphite; ceramics and ceramic fibers including calcium silicate; brick and cement; plastics and polymers including polyisocyanurate, polyurethane, polystyrene, and elastomers; natural materials including perlite, vermiculite, mineral wool, cork, sawdust, and woodshavings; and any combination thereof. Suitable structures of insulation 222 may include, but not be limited to, beads; honeycomb; porous or nanoporous; aerogel; foam including premade or foamed in place; fibers; fabric; wool; sheet; rigid board; and any combination thereof. In some embodiments, insulation 222 or a component thereof may comprise a reflective surface, e.g., foil-faced, and/or an absorbative surface. One skilled in the art, with the benefit of this disclosure should understand the plurality of insulation materials and configurations within apparatus 200 including layering available to achieve the desired function of insulating given the operational requirements like temperature and size.

Test cell 210 is generally an enclosure comprising at least one wall and at least one sealable opening. In some embodiments, test cell 210 may be removable from apparatus 200. The general configuration of test cell 210 may be any configuration suitable for a desired experiment. Suitable shapes include, but are not limited to, cylindrical, spherical, ellipsoidal, polyagonal, and the like, and any hybrid thereof. Generally, but not always, the length of test cell 210 is longer than its width. Test cell 210 should be configured to accept a sample, typically through a sealable opening. Such configurations may incorporate, by way of nonlimiting examples, a door, a hatch, a port, and the like, and any combination thereof. One skilled in the art would understand that the sealable opening should be configured to be compatible with operational conditions like temperature, pressure, and sample composition. Further, one skilled in the art should understand that the physical configuration of test cell 210 should allow for the operation of test cell 210 in apparatus 200 under selected operation parameters including, but not limited to, those outlined herein of removability, movement, operability with other elements of apparatus 200, operability with external elements to apparatus 200, and any combination thereof.

It should be noted that when “about” is provided at the beginning of a numerical list, “about” modifies each number of the numerical list. It should be noted that in some numerical listings of ranges, some lower limits listed may be greater than some upper limits listed. One skilled in the art will recognize that the selected subset will require the selection of an upper limit in excess of the selected lower limit.

Test cell 210 may be designed to contain elevated or reduced pressures therein. Test cell 210 may be designed to hold pressures ranging from a lower limit of about 1 psi, 10 psi, 100 psi, 500 psi, or 1000 psi to an upper limit of about 10,000 psi, 7500 psi, 5000 psi, 2500 psi, or 1000 psi, and wherein the pressure may range from any lower limit to any upper limit and encompass any subset therebetween.

Test cell 210 may be made of any known material that is compatible with the sample, compatible with any operational condition like temperature and pressure, and provide the necessary thermal transmission to change the temperature of the sample at a desired rate. Suitable materials for test cell 210 may include, but not be limited to, metals and metal alloys like aluminum, copper, brass, chrome, and stainless steel; plastics and polymers; glass; composites including those with additives like carbon fibers and nanomaterials; and any combination thereof. In some embodiments, test cell 210 may comprise a coating on at least a portion of the internal wall(s). In some embodiments, said coating may assist in sample compatibility. It should be noted that “a coating,” as used herein, is a general term that includes a liner, a film, a skin, a cover, a crust, a glaze, a laminate, a paint, a membrane, and the like. Further, “a coating” may be permanent, semi-permanent, removable, disposable, and/or degradable. Moreover, “a coating” may be solid like polymer or metal or liquid like grease.

In some embodiments, test cell 210 may be of a configuration already in use by one skilled in the art, i.e., apparatus 200 may be designed to accept known configurations of test cell 210.

Thermal element 240 may be any known heating and/or cooling element including, but not limited to, an electric heater, an infrared heater, a thermoelectric cooler, and any combination thereof. Suitable configuration for thermal element 240 may include, but not be limited to, coils, plates, strips, finned strips, and the like, and any combination thereof. Having thermal element 240 and test cell 210 close to each other, especially if test cell 210 is rotating, may advantageously allow for more efficient thermal conduction therebetween. In some embodiments, thermal element 240 and test cell 210 may be relationally configured to be separated by a distance of about 5 mm or less. In some embodiments, thermal element 240 and test cell 210 may be relationally configured to be in physical contact in at least one point. In some embodiments, thermal element 240 and test cell 210 may be relationally configured to be separated by a distance ranging from a lower limit of physical contact, about 1 mm, about 2 mm, about 5 mm, or about 10 mm to an upper limit of about 10 cm, 5 cm, 25 mm, 20 mm, 15 mm, or 10 mm, and wherein the distance may range from any lower limit to any upper limit and encompass any subset therebetween. It should be noted, that the upper limit of the distance between thermal element 240 and test cell 210 is when thermal element 240 is no longer in thermal communication with test cell 210, which may be in excess of 10 cm.

One skilled in the art, with the benefit of this disclosure, should understand how to make thermal element 240 operable to heat and/or cool test cell 210. The range of temperatures apparatus 200 may be operated at may depend on the type and configuration of thermal element 240, and therefore is limited only by hardware and hardware configuration. In some embodiments, thermal element 240 may be capable of causing test cell 210 to reach and/or maintain temperatures ranging from a lower limit of about −150° F., −100° F., 0° F., 50° F., 100° F., 200° F., 300° F., or 400° F. to an upper limit of about 900° F., 800° F., 700° F., 600° F., 500° F., or 400° F., and wherein the temperature may range from any lower limit to any upper limit and encompass any subset therebetween.

Further, one skilled in the art, with the benefit of this disclosure, should understand the plurality of thermal element 240 options both in type and configuration. Further, one skilled in the art should understand the plurality of physical configuration applicable to operably integrating thermal element 240 into apparatus 200 including, but not limited to, about test cell 210, near test cell 210, proximal to test cell 210, in contact with test cell 210, integrated as part of about test cell 210, and any combination thereof.

Referring again to FIG. 2, in some embodiments, apparatus 200 may comprise shaft 250 operably connected to test cell 210 and extending parallel to cell central axis 214 from test cell 210 to drive connection 252, e.g., a cog, which itself is operably connected to shaft 250. As used herein, the term “cog” includes similarly operating elements that may be toothed and/or lipped including, but not limited to, a pulley, a gear, a gear wheel, a geared wheel, a cogwheel, and a sprocket. In some embodiments, shaft 250 may be more than one piece wherein said pieces are operably connected into shaft 250. In some embodiments, shaft 250 may comprise bearings 254 disposed about shaft 250 between test cell 210 and drive connection 252. It should be noted that, as used herein, disposed may include completely around, on opposing sides, space around, equally spaced around, randomly space around, and the like.

Suitable drive connections may include direct connections to a driving mechanism and/or a component, e.g., a cog, capable of operably connecting to a drive mechanism. Suitable drive mechanisms may include, but not be limited to, motors, direct drive motors, pancake motors, stepper motors, bushed DC motors, bushless DC motors, permanent magnetic synchronous motors, AC induction motors, switched reluctance motors, electrostatic motors, hydraulic motors, pneumatic motors, heat engines, and the like. As used herein, the term “motor” means a machine or system designed to convert energy into useful motions and includes engines.

In some embodiments, driving mechanism 270 (not shown in FIG. 2) may be operably connected to drive connection 252. In some embodiments, driving mechanism 270 may be an element of apparatus 200. In some embodiments, driving mechanism 270 may be a separate element that may be operably connected to drive connection 252 of apparatus 200. In some embodiments, driving mechanism 270 may be external to housing 220.

In some embodiments, driving mechanism 270 may cause drive connection 252, shaft 250, and test cell 210 to be manipulated. Manipulations of test cell 210 may include, but not be limited to, 360° rotation about cell central axis 214; rocking about cell central axis 214, i.e., rotating in one direction about cell central axis 214 for less than about 360° then rotating in the other direction about cell central axis 214 for less than 360° and repeating; moving back and forth along cell central axis 214; and any combination thereof.

One skilled in the art would recognize the plurality of ways to operably connect drive connection 252 and driving mechanism 270. In some embodiments, drive connection 252 and driving mechanism 270 may be operably connected by connection element 256. Suitable connection elements may include, but not be limited to, a belt (as shown in FIG. 7), a rubber band, a chain, a cog belt, a sprocket, a shaft, a bar, and any combination thereof. By way of nonlimiting example shown in FIG. 7, driving mechanism 270 may be operably connected to drive connection 272 which itself is operably connected to drive connection 252 by a belt connection element 256. It should be noted that drive connection 272 and drive connection 252 need not be the same size. One skilled in the art should understand that the size and shape of drive connection 252 may effect the movement parameters, including speed, of shaft 250 and ultimately test cell 210.

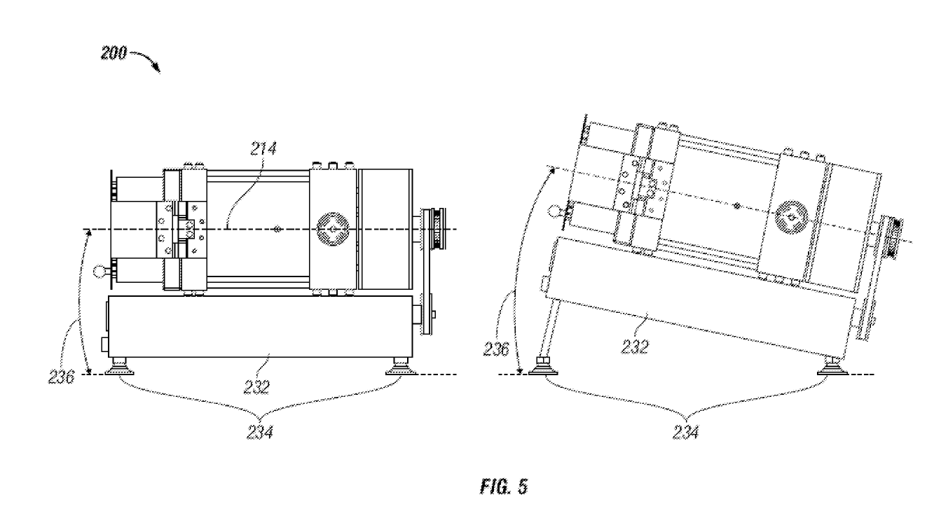

Drive connection 252 may be made of any known material capable of withstanding operations stresses. Suitable materials include, but are not limited to, metals and metal alloys like aluminum, copper, brass, chrome, chrome steel, carbon alloy steel, and stainless steel; ceramics including silicon nitride; plastics and polymers like polyether ether ketone (PEEK), polyurethane, and polytetrafluoroethylene (PTFE); composites including those with additives like carbon fibers, glass fibers, and nanomaterials; fiberglass; and any combination thereof. Drive connection 252 may be of any known configuration suitable for at least the operations described herein including, but not limited to, circular (shown in FIGS. 5-8) and substantially spherical.

Bearings 254 may be any known bearing type including, but not limited to, ball bearings, roller bearings, roller thrust bearings, tapered roller bearing, and any combination thereof. Bearings 254 may be made of any known material capable of withstanding the operations stress. Suitable materials include, but are not limited to, metals and metal alloys like aluminum, copper, brass, chrome, chrome steel, carbon alloy steel, and stainless steel; ceramics including silicon nitride; plastics and polymers like polyether ether ketone (PEEK), polyurethane, and polytetrafluoroethylene (PTFE); composites including those with additives like carbon fibers, glass fibers, and nanomaterials; fiberglass; graphite; any hybrid thereof; any mixture thereof; and any combination thereof. One skilled in the art, with the benefit of this disclosure, should understand other mechanical elements that may replace bearings 254 to achieve the same operability, including, but not limited to, bushings.

Suitable materials for shaft 250 may include any known material that can endure the mechanical stresses of operation disclosed herein. Suitable materials include, but are not limited to, metals and metal alloys like aluminum, copper, brass, chrome, chrome steel, carbon alloy steel, and stainless steel; ceramics including silicon nitride; plastics and polymers like polyether ether ketone (PEEK); composites including those with additives like carbon fibers, glass fibers, and nanomaterials; fiberglass; ceramics; and any combination thereof. One skilled in the art should understand, given the configuration of shaft 250 operably connecting test cell 210 and drive connection 252, that opposing ends of shaft 250 may need to be at very different temperatures depending on the operating temperature of test cell 210 and material of drive connection 252. Therefore, the material of shaft 250 should be chosen accordingly.

Referring again to FIG. 2, in some embodiments, apparatus 200 may comprise thermal dam 260 disposed between test cell 210 and drive connection 252. In some embodiments, thermal dam 260 may be connected to shaft 250. In some embodiments, shaft 250 may be configured to comprise thermal dam 260. By way of nonlimiting example, shaft 250 may be three pieces including a first piece operably connecting the cell to the second piece being the thermal dam, the second piece operably connecting the first piece to the third piece, and the third piece operably connecting the second piece to drive connection 252. In some embodiments, thermal dam 260 may be proximal to shaft 250. In some embodiment, thermal dam 260 may be disposed about shaft 250. In some embodiments where shaft 250 comprises bearings 254, bearings 254 may be disposed between thermal dam 260 and drive connection 252 and/or disposed between test cell 210 and thermal dam 260, whether thermal dam 260 is part of the shaft or otherwise. In some embodiments where shaft 250 comprises bearings 254 disposed between thermal dam 260 and drive connection 252, thermal dam 260 may advantageously extend the lifetime of bearings 254 by reducing the temperature to which bearings 254 are exposed.

Thermal dam 260 may be designed to interrupt the flow of heat along shaft 250 from test cell 210 toward drive connection 252. In some embodiments, apparatus 200 may comprise at least one fan 262 in fluid communication with thermal dam 260. Fan 262 may operate to reduce thermal transfer via the air around shaft 250 and/or thermal dam 260 by transporting gas toward or away from thermal dam 260. Further, one skilled in the art, with the benefit of this disclosure, would understand that thermal dam 260 may be structurally designed to provide a longer path for heat to traverse by comprising two plate with diameters larger than shaft 250 and connected at, or close to the edges.

Suitable materials for thermal dam 260 include all materials with adequate thermal conductivity to interrupt heat transfer along shaft 250 from test cell 210 toward drive connection 252. It should be noted that in some embodiments, thermal dam 260 may be a vessel for holding beads, fluids, dusts, particles, and the like of any of the above materials. One skilled in the art, with the benefit of this disclosure, would understand that the material should be compatible with the temperatures in which apparatus 200 will operate, e.g., at operating temperatures in excess of about 400° F. a thermal dam comprising woods may not be appropriate. Examples of suitable materials that may be at least a portion of thermal dam 260 include, but are not limited to, aluminum; aluminum alloys including 1050A, 6061, and 6063; brass; lead; stainless steel; sandstone; concrete; rock; volcanic minerals; pozzolans; epoxy; cement; rubber; mineral oil; polyethylene; polypropylene; polyurethanes; polystyrenes; polyvinyl chlorides; polytetrafluoroethylene; hollow fiber insulation; woods; sawdust; aerogels; bitumen; graphite; carbon fiber composites; ceramics; silicas; aluminas; cork; fiberglass; glass; pyrex; quartz; granite; gypsum; marble; mica; plaster; foamed plastics; materials with thermal conductivity coefficients less than about 150 W/(m*K) at 25° C.; any mixture thereof; and any combination thereof.

Referring again to FIG. 2, in some embodiments, apparatus 200 may comprise cell bushings 216 disposed inline with or radially from cell central axis 214 such that test cell 210 is disposed between bushings 216 and shaft 250 and bushings 216 are disposed between insulation 222 and test cell 210. Bushing 216 may operably work within apparatus 200 to stabilize test cell 210 while being manipulated, especially while rotating or rocking. Suitable materials for said bushing may be any known material suitable for bushings including, but not limited to, metals and metal alloys like brass; plastics and polymers like polyether ether ketone (PEEK), polyurethane, and polytetrafluoroethylene (PTFE); composites including those with additives like carbon fibers, glass fibers, and nanomaterials; carbon nanotube carpets; and any combination thereof. Bushing 216 may operably work within apparatus 200 in conjunction with a sensor, e.g., a thermocouple, when electrically isolated from test cell 210.

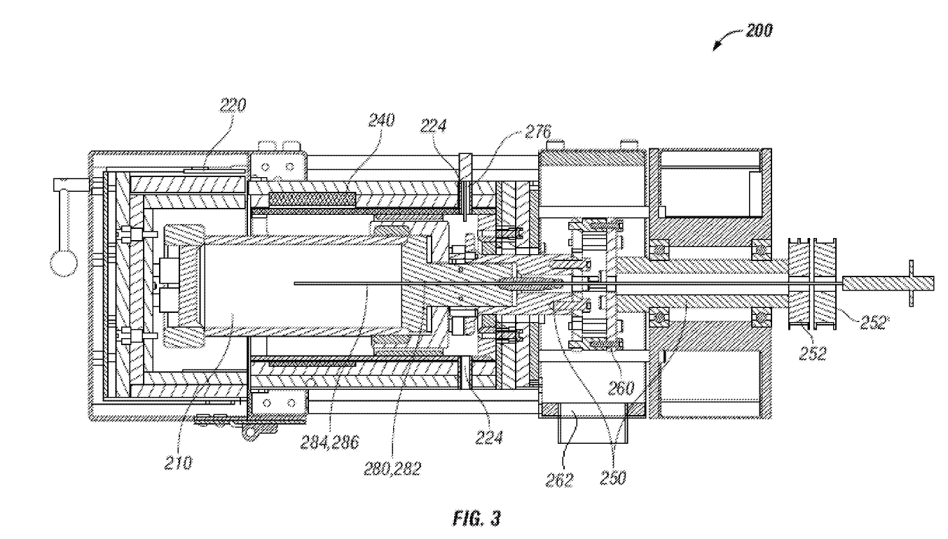

Referring now to FIG. 3, in some situations, it my be desirable to monitor various properties and/or parameters of apparatus 200. In some embodiments, apparatus 200 may comprise at least one sensor 276. Suitable properties and parameters of apparatus 200 to sense and/or measure include, but are not limited to, thermal element 240 temperature; test cell 210 temperature; housing 220 temperature; shaft 250 temperature; thermal dam 260 temperature; fan 262 speed; test cell 210 movement direction and/or speed; shaft 250 movement direction and/or speed; and any combination thereof. In some embodiments, sensor 276 may operate wirelessly. In some embodiments, housing port 224 may be used to house at least part of sensor 276 and/or its corresponding connections. By way of nonlimiting example as shown in FIG. 3, housing port 224 may contain at least a portion of sensor 276, which may be a thermocouple, that extends from outside housing 220 to inside housing 220 to a point near test cell 210.

Referring again to FIG. 3, in some situations, it may be desirable to monitor and/or affect various properties of a sample in test cell 210 while apparatus 200 is in operation. In some embodiments, test cell 210 may comprise at least one cell sensor port 280 that extends from outside test cell 210 to inside test cell 210. One skilled in the art would understand the plurality of ways to design cell sensor port 280. Suitable configurations may include those that allow for cell sensor port 280 to contain at least a portion of at least one cell sensor 284 and/or corresponding connections. In some embodiments, test cell 210 may comprise cell sensor port 280 for multiple sensors, cell sensor port 280 for single sensors, and any combination thereof. One skilled in the art, with the benefit of this disclosure, should understand that such cell sensor port 280 should be configured such that materials do not undesirably pass therethrough, e.g., leak sample or loose pressure.

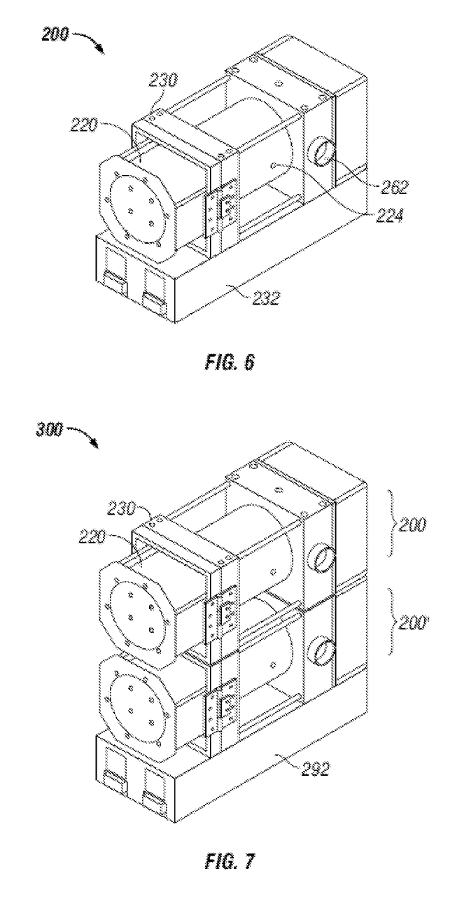

Suitable sample properties to monitor, sense, measure, affect, actuate, and/or stimulate may include, but not be limited to, sample temperature, pressure within test cell 210, sample conductivity, sample composition, sample turbidity, sample density, sample rheology, particle size distribution, emulsion stability, and any combination thereof. Suitable sensors for measuring, monitoring, and/or sensing sample properties may include, but not be limited to, thermal sensors like thermocouples; conductivity sensors; spectroscopic sensors including those for measuring fluorescence, absorbance, FT-IR, and Raman; pressure sensors; optical computing devices like an integrated computational element (ICE), which separates electromagnetic radiation related to the characteristic or analyte of interest from electromagnetic radiation related to other components of a sample; multimodal sensors; and any combination thereof. Further details regarding how the optical computing devices can separate and process electromagnetic radiation related to the characteristic or analyte of interest are described in U.S. Pat. No. 7,920,258, the entire disclosure of which is incorporated herein by reference. By way of nonlimiting example shown in FIG. 6, cell sensor port 280 with a thermocouple as cell sensor 284 configured such that test cell 210 can rotate and/or rock about cell central axis 214. One skilled in the art, with the benefit of this disclosure, should understand that additional configuration adjustments may be required for cell sensor 284 and/or corresponding connections to accommodate movement of test cell 210. Further, one skilled in the art should understand that cell sensor 284 should be compatible with the sample and operational requirements like temperature and pressure.

In some embodiments, it may be desirable to affect, actuate, and/or stimulate the sample with a stimuli. Examples of stimuli may include, but not be limited to, structures within test cell 210 to change the movement of the sample, introduction of materials to change the composition of the sample, introduction of materials to challenge the composition of a sample (e.g., testing the pH limitations of a foamed treatment fluid at elevated temperatures), introduction of an electrical stimuli, introduction of an acoustic stimuli, introduction of a vibration stimuli, change of the pressure (increase or decrease) in test cell 210, or any combination thereof.

In some embodiments, test cell 210 may be designed to include a structure to affect the sample movement therein. Affecting sample movement within test cell 210 may be to change the fluid mixing dynamics and/or to measure a fluid property like viscosity. In some embodiments, a structure (not shown) within test cell 210 may be used in conjunction with cell sensor 284 including, but not limited to, for the purposes of measuring viscosity and/or turbidity. Suitable structures that may be included within test cell 210 include, but are not limited to, a vane; a plate oriented with its thickness along cell central axis 214; a web of intertwined rods that may or may not be curved; unconnected spokes, fins, or blades attached internally to test cell wall 212; a cylinder oriented concentrically within test cell 210 that may or may not be attached to test cell wall 212; and any combination thereof. In some embodiments, a structure for affecting sample movement may be stationary, or substantially stationary, while test cell 210 moves. By way of nonlimiting example, a plurality of fins may be pneumatically controlled to change orientation within test cell 210. Further, by way of nonlimiting example, a cylinder oriented concentrically within test cell 210 may comprise magnets that correspond to magnets outside test cell 210 within housing 220 proximal to test cell 210 and bearings to allow for test cell 210 to move relative to the cylinder. Further the bearings may provide a defined spacing between the cylinder and test cell 210. One skilled in the art would understand that structures to be included within test cell 210 should be made of materials that do not significantly react with a sample and can withstand the operation requirements like temperature.

Referring again to FIG. 3, in some situations, it may be desirable to add or remove materials from the sample in test cell 210 while apparatus 200 is in operation. Suitable materials to be transported include, but are not limited to, fluids (gas and/or liquid), solids, or any combination thereof. In some embodiments, transport of a gas may yield or may be to effect a pressure increase or decrease within test cell 210. In some embodiments, test cell 210 may comprise at least one cell material port 282 that extends from outside test cell 210 to inside test cell 210. One skilled in the art would understand the plurality of ways to design cell material port 282. Suitable configurations may include those that allow for transporting of materials into and/or out of test cell 210 through material transport element 286. By way of nonlimiting example shown in FIG. 6, cell material port 282 with a fluid injector as material transport element 286 is configured such that test cell 210 can rotate and/or rock about cell central axis 214. One skilled in the art with the benefit of this disclosure, should understand that additional configuration adjustments may be required for material transport element 286 and/or corresponding connections to accommodate movement of test cell 210. Further, one skilled in the art, with the benefit of this disclosure, should understand that material transport element 286 should be configured and be made of appropriate materials to effectively operate within the temperature and pressure ranges desired.

In some embodiments, cell sensor port 280 and cell material port 282 may be one in the same, i.e., a single port may be configured to accommodate both at least cell sensor 284 and at least one material transport element 286 and/or a single port may be configured to accommodate at least cell sensor 284 or at least one material transport element 286. One skilled in the art should understand that cell sensor port 280 and cell material port 282 should be appropriately configured such that if capable of being empty they may be plugged so as to maintain the necessary enclosure of test cell 210 within the operation requirements like temperature and pressure.

It should be noted that cell sensor port 280 and/or cell material port 282 may be configured to monitor, sense, measure, affect, actuate, and/or stimulate the sample in the ways described herein. The descriptive terms of cell sensor port 280 and/or cell material port 282 should not be considered limiting as to the function and/or capabilities of the ports. Further it should be noted that one skilled in the art, with the benefit of this disclosure, should understand how to configure cell sensor port 280 and/or cell material port 282 to accommodate an element necessary to achieve the desired monitoring, sensing, measurement, affect, actuation, and/or stimulation. One skilled in the art should further understand the element may be limited by the physical limitations of some embodiments of configurations of apparatus 200 and/or the desired operational conditions of apparatus 200.

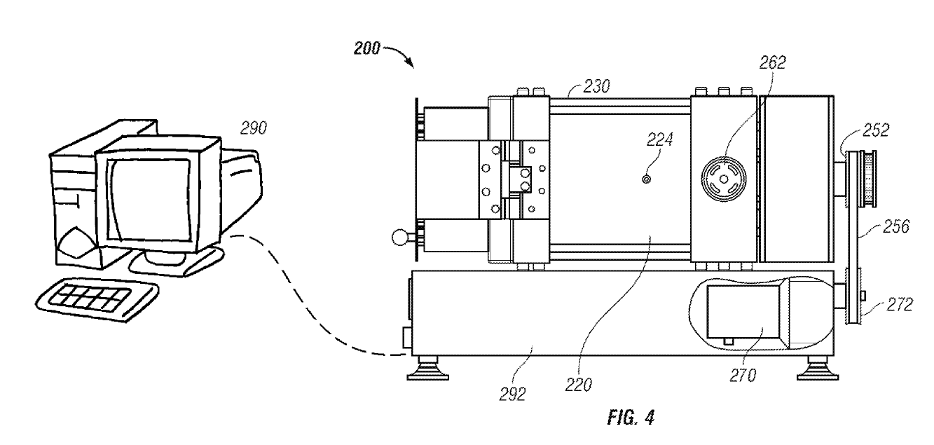

In some embodiments, apparatus 200 may comprise control mechanism 290, a nonlimiting configuration of which is shown in FIG. 4 as a computer. As used herein, the term “control system” refers to a system that can operate to receive and send electronic signals and may include functions of interfacing with a user, providing data readouts, collecting data, storing data, changing variable setpoints, maintaining setpoints, programming experimental parameters, providing notifications of failures and/or test interruptions, and any combination thereof. In some embodiments, control mechanism 290 may be an element of apparatus 200. In some embodiments, control mechanism 290 may be a separate element that may be operably connected to elements of apparatus 200 including, but not limited to, sensor 276, thermal element 240, fan 262, driving mechanism 270, cell sensor 284, material transport element 286, and any combination thereof. In some embodiments, control mechanism 290 may be external to housing 220. In some embodiments, cell sensor 284 and/or material transport element 286 may be operably connected to a control mechanism. Suitable control mechanisms 290 include, but are not limited to, variable transformers, ohmmeters, programmable logic controllers, digital logic circuits, electrical relays, computers, and any combination thereof. In some embodiments, control mechanisms 290 may be further capable of storing information from the functions listed above, both inputs and outputs.

Referring now to FIG. 4, in some embodiments, apparatus 200 may comprise base 232. In some embodiments, base 232 may house elements of apparatus 200 including, but not limited to, control mechanism 290, driving mechanism 270, a pressurization system (not shown), and any combination thereof.

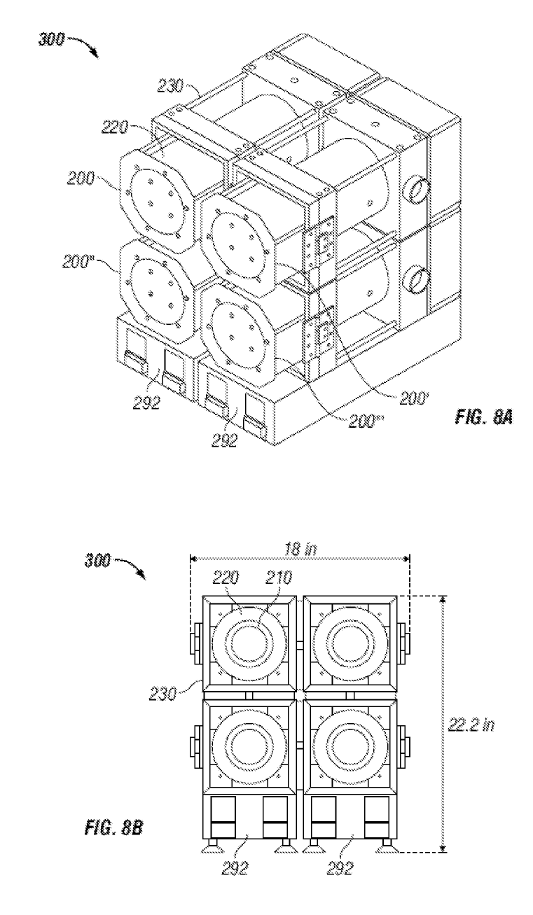

Referring now to FIG. 5, in some embodiments, base 232 may comprise at least one leg 234 including, but not limited to one, two, three, four, five, six, and so on. One skilled in the art would understand that leg 234 may have a plurality of configurations and not all legs 234 must be of the same configurations. In some embodiments, base 232 may comprise at least one leg 234 capable of extending. In some embodiments, some or all legs 234 may extend so as to level apparatus 200. In some embodiments, extending and/or retracting some or all legs 234 may cause angle 236 between cell central axis 214 and the ground to change, as shown in FIG. 8. Angle 236 may vary from any angle ranging from 0° to about 90°. One skilled in the art would understand that the configuration of apparatus 200 should be taken into consideration if wishing to accommodate larger angles. One skilled in the art would understand the plurality of mechanisms by which leg 234 may be extended. In some embodiments, leg 234 may be extended hydraulically, in some embodiments, leg 234 may be extended or retracted from a signal from control mechanism 290. In some embodiments, leg 234 operably connected to control mechanism 290 may be programmed to extend and retract in a cyclical manner.

Referring now to FIG. 6, in some embodiments, apparatus 200 may comprise frame 230 that houses at least a portion of housing 220. In some embodiments, frame 230 may be a separate element that may be operable to house at least a portion of housing 220 of apparatus 200. In some embodiments, frame 230 may be configured to connect to base 232. In some embodiments, frame 230 may be configured to comprise legs 234, which at least one may optionally be extendable as described above.

Referring now to FIG. 5, in some embodiments, frame 230 may be configured such that multiple apparatuses 200 may be stacked vertically, horizontally, or any combination thereof. One skilled in the art, with the benefit of this disclosure, should understand the plurality of dimensional configurations in which the various elements and components of apparatus 200 can be configured and that any dimensions provided in the figures are nonlimiting embodiments. Further, one skilled in the art should recognize the scalability of apparatus 200.

Referring now to FIGS. 7 and 8, in some embodiments, apparatus 200 may be part of modular system 300. In some embodiments, modular system 300 may comprise at least one apparatus 200, including, but not limited to, one, two (FIG. 7), three, four (FIG. 8), five, six, seven, and so on. In some embodiments, apparatus 200 of modular system 300 may be according to any embodiments disclosed herein. In modular system 300 embodiments with at least two apparatuses 200, apparatuses 200 may be of the same configuration, of different configurations, or any combination thereof. In some embodiments, modular system 300 comprising at least two apparatuses 200 may be controlled, monitored, manipulated, operably connected to, etc. in any combination with any combination of components including, but not limited to, base 232, legs 234, connection element 256, fan 262, driving mechanism 270, drive connection 272, sensor 276, cell sensor 284, cell material transport 282, control mechanism 290, control box 292, and any combination thereof.

In modular system 300 embodiments with at least two apparatuses 200′, 200″, and so on, apparatuses 200′, 200″, and so on may be operably connected to a single driving mechanism 270 through their respective cogs 252′, 252″, and so on (not shown). By way of nonlimiting example, first apparatus 200′ may comprise two cogs 252′ in series and second apparatus 200″ may comprise a single drive connection 252″. One drive connection 252′ of apparatus 200′ may be operably connected to driving mechanism 270 while the other drive connection 252′ of apparatus 200′ may be operably connected to the single drive connection 252″ of second apparatus 252″. This may allow for driving mechanism 270 to operate both apparatuses 200′, 200″ of modular system 300 simultaneously. Further, one skilled in the art would understand that the speed of movement imparted on each drive connection may be different by changing the size of each drive connection appropriately. One skilled in the art would recognized that the orientation of apparatus 200′ and apparatus 200″ may be any configuration such that they can be operably connected including, but not limited to, stacked, arranged horizontally, arranged back-to-back, arranged diagonally, and the like.

Operable connections between two or more drive connections 252 may be achieved by any known connection element 256 including, but not limited to, those disclosed herein for an operably connect of driving mechanism 270 to drive connection 252. In some embodiments, the operable connection between driving mechanism 270 and drive connection 252 may be different than that between tow drive connections 252 within modular system 300. In some embodiments, connection element 256 may include a bar, or the like, operably connected to driving mechanism 270 with said bar being operably connected to drive connection 252 of each apparatus 200.

In some embodiments, elements and components described above may be a part of modular system 300 as opposed to apparatus 200 including, but not limited to, frame 230, base 232, legs 234, connection element 256, fan 262, driving mechanism 270, drive connection 272, sensor 276, control mechanism 290, control box 292, and any combination thereof. That is to say, the minimum elements and components of apparatus 200 may include test cell 210, housing 220, insulation 222, thermal element 240, shaft 250, and drive connection 252. Optional elements and components that may be of apparatus 200 without overlap with system 300 elements and components include cell bushings 216, housing port 224, bearings 254, thermal dam 260, cell sensor port 280, and cell material port 282. Further, some elements and components may be separate from both modular system 300 and apparatus 200 and may be appropriately operable connected to modular system 300 and/or apparatus 200, including, but not limited to, base 232, legs 234, connection element 256, fan 262, driving mechanism 270, drive connection 272, sensor 276, cell sensor 284, cell material port 282, control mechanism 290, control box 292, and any combination thereof.

The present invention provides for apparatus 200 and/or system 300 to be used in a variety of methods, only some of which are included herein. The methods may advantageously simulate conditions that cannot be otherwise simulated with traditional roller ovens. Apparatus 200 and/or system 300 also provide for methods that allow real-time monitoring and manipulation of a sample which they are testing. Further, apparatus 200 and/or system 300 allow for integration into quality control methods that may be practiced in a laboratory setting and/or in the field.

In some embodiments, apparatus 200 or system 300 according to a disclosed embodiment may be used to test a sample in test cell 210. In some embodiments, samples may be a fluid including, but not limited to, gases, liquids, fluids comprising solids, fluids that harden, gelled fluids, foamed fluids, or any combination thereof. In some embodiments, a sample may be a fluid or portion of a fluid provided, produced in a laboratory, produced by a manufacturing process, produced at a wellbore site, or provided at a wellbore site. Examples of fluids may include, but not be limited to, treatment fluids, drilling fluids, drill-in fluids, completion fluids, workover fluids, lost circulation fluids, fracturing fluids, acidizing fluids, wellbore strengthening fluids, packer fluids, spacer fluids, cementitious slurries, insulation fluids, and the like.

In some embodiments, methods of testing a sample may include, but not be limited to, manipulating the cell, changing the sample temperature, introducing materials, changing the pressure within the cell, changing the manipulation of the cell, or any combination thereof. It should be noted that manipulating the cell is not required, i.e., apparatus 200 and/or system 300 may be used for static testing of a sample. Further, apparatus 200 and/or system 300 may be used with both the static and dynamic movement in a single test.

In some embodiments, the sample may be analyzed before a test, during a test, after a test, or any combination thereof. Analysis may include, but not be limited to, chemical analysis, physical analysis, thermal analysis, electrical analysis, turbidity analysis, rheological analysis, density (including density gradient) analysis, particle size distribution analysis, and any combination thereof. The physical and/or chemical properties tested may include, but not be limited to, chemical composition including production of byproducts and/or degradation of the sample; physical make up like settling of particulates or breaking of foams or gels; thermal profile of the sample including points of endothermic or exothermic reactions; electrical conductivity of a sample; viscosity of a sample; or any combination thereof.

In some embodiments, analysis may be done on-line, off-line, or a combination thereof relative to the test. In some embodiments, off-line analysis may be performed during a test when a portion of the sample is taken during the test. In some embodiments, on-line analysis may be performed by sensor 276 and/or cell sensor 284. In some embodiments, on-line analysis may be performed by extracting a portion of the sample during the test through cell sensor port 280 wherein cell sensor port 280 is operably connected to another instrument like a gas chromatograph, mass spectrometer, a UV-visible spectrometer, a fluorometer, the like, or any combination thereof. In some embodiments, on-line analysis may be conducted in conjunction with a computer operably connected to apparatus 200 and/or system 300.

In some embodiments, operational conditions may be adjusted during a test. In some embodiments, operation conditions may be adjusted in response to analysis during a test. In some embodiments, operational conditions that may be changed include, but are not limited to, temperature of the sample; manipulation of test cell 210, including speed of manipulation and/or type of manipulation; pressure within test cell 210; composition of the sample; or any combination thereof.

In some embodiments, control mechanism 290 may be used in conjunction with monitoring and/or changing operation conditions. In some embodiments, control mechanism 290 may be a computer that may allow for remote operation and/or monitoring (e.g., via the internet), for programmed operation, for logging of test parameters and results, for logging of apparatus history and/or errors, for programmed calibration procedures, or any combination thereof.

In some embodiments, a sample may comprise a liquid, a gas, a solid, or any combination thereof. Suitable samples may include, but not be limited to, any composition that may be placed in a subterranean formation. Nonlimiting examples of suitable sample may include treatment fluids, foamed treatment fluids, treatment fluids comprising particulates, components of a treatment fluid, solid particulates and/or beads, the solids of a cementitious compositions, slurried cementitious compositions, components of a downhole tool, and the like.

In some embodiments, based on the analysis of a sample, a fluid composition may be changed including, but not limited to, the fluid from which the sample was taken, a second fluid, or any combination thereof. In some embodiments, the second fluid may be a fluid to be produced and/or an existing fluid. In some embodiments, changing a second fluid may include changing a fluid-additive composition that may be used to produce the second fluid. The fluid-additive composition may be a fluid itself, a solid, a mixture of solids, or a combination thereof. In some embodiments, the fluid to which the composition has been changed may be introduced into a wellbore penetrating a subterranean formation.

In some embodiments, based on the analysis of a sample, a composition of solids may be changed including, but not limited to, the solids from which the sample was produced, a second composition of solids, or any combination thereof. In some embodiments, the second composition of solids may be solid to be produced and/or existing solids. In some embodiments, changing a second composition of solids may include changing an additive composition that may be used to produce the second composition of solids. In some embodiments, changing a second composition of solids may include changing the ratios of the solids that make up the second composition of solids. In some embodiments, the second composition of solids of which the composition has been changed may be introduced into a wellbore penetrating a subterranean formation in solid form, as part of a fluid, or any combination thereof.

Of the many advantages of the present invention, portability of apparatus 200 and/or system 300 provides for use at a wellbore site or a laboratory near a wellbore site.

Further, advantageously apparatus 200 and/or system 300 may be used to simulate conditions a fluid may experience not only within a subterranean formation, but also during transport. By way of nonlimiting example, a fluid may be tested under conditions that simulate transport to an offshore well site. Conditions a fluid may see in such transport includes rocking motions and temperature fluctuations.

In another advantage of apparatus 200 and/or system 300 over traditional roller ovens may be the applicability to test samples like cements in new ways. By way of nonlimiting example, a cementitious sample, like a cement slurry, may be analyzed in situ for the exothermic reaction at the point of setting while increasing temperature and manipulating test cell 210 continuously in 360°. Such an analysis may provide insight into the behavior of cementitious fluid while being pumped in a wellbore.

In some embodiments, a housing may include a test cell being enclosed and having at least one test cell wall and one sealable opening and has a cell central axis defined along the center line of the length of the test cell, a thermal element in thermal communication with the test cell, and an insulation, at least a portion of the insulation being disposed about the test cell and the thermal element. In some embodiments, an apparatus may generally include a housing, a drive connection, and a shaft operably connected to the test cell and extending parallel to the cell central axis from the test cell through the insulation to the drive connection to which the shaft is operably connected. In some embodiments of the present invention, a modular system may include at least one apparatus and a frame that houses at least a portion of the housing, and a driving mechanism operably connected to the drive connection.

Some embodiments may involve testing a sample in the test cell of an apparatus operably connected to a driving mechanism by at least manipulating the test cell and analyzing the sample.

Some embodiments may involve testing the stability of deep-sea treatment fluids by testing a sample in the test cell of an apparatus operably connected to a driving mechanism by at least manipulating the test cell, changing the sample temperature to a first temperature below room temperature, changing the sample temperature to a second temperature above room temperature, and analyzing the sample.

Some embodiments may involve testing a cement composition by testing a cementitious sample in the test cell of an apparatus operably connected to a driving mechanism by at least manipulating the test cell; changing the sample temperature; and monitoring the temperature within the test cell.

While the disclosure herein is drawn toward the subterranean operation industry, one skilled in the art, with the benefit of this disclosure, should recognize the parallel applications like food and beverage, automotive and motor fluids, and lubricants.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces, if there is any conflict in the usages of a word or term in this specification and one or more patent or other documents that may be incorporated herein by reference, the definitions that are consistent with this specification should be adopted.

The invention claimed is:

1. A method comprising:
providing an apparatus that comprises:
a housing that comprises
a test cell being enclosed and having at least one test cell wall and one sealable opening and has a cell central axis defined along the center line of the length of the test cell,
a thermal element in thermal communication with the test cell, and
an insulation, at least a portion of the insulation being disposed about the test cell and the thermal element;
a drive connection; and
a shaft operably connected to the test cell and extending parallel to the cell central axis from the test cell through the insulation to the drive connection to which the shaft is operably connected;
providing a driving mechanism operably connected to the drive connection;
providing a sample in the test cell of the apparatus;
manipulating the test cell; and
analyzing the sample.

2. The method of claim 1 further comprising:
changing the pressure within the test cell.

3. The method of claim 1, wherein manipulating the test cell involves an action selected from the group consisting of 360° rotation about cell central axis; rocking about cell central axis; moving back and forth along cell central axis; and any combination thereof.

4. The method of claim 1, wherein manipulating the test cell involves changing the sample temperature.

5. The method of claim 1 further comprising:
allowing at least one control mechanism connected to an attachment of the apparatus to function in a way selected from the group consisting of collecting data from the attachment, sending a signal to the attachment, and any combination thereof,
wherein the attachment selected from the group consisting of a housing thermal sensor in a housing sensor port that extends from outside the housing to inside the housing near the test cell; a test cell sensor in a test cell sensor port; a fluid transfer device operably connected to a test cell fluid port; and any combination thereof.

6. The method of claim 5, wherein the control mechanism is a computer.

7. The method of claim 1, wherein the thermal element and the driving mechanism are operably connected to at least one control mechanism.

8. The method of claim 7, wherein the control mechanism is a computer capable of running an automated procedure for changing the temperature and moving the test cell.

9. The method of claim 1, wherein the apparatus is located at a well site.

10. The method of claim 1 further comprising:
changing the fluid composition based on the analysis.

11. The method of claim 1 further comprising:
changing a fluid-additive composition used to produce a second fluid based on the analysis.

12. The method of claim 1 further comprising:
changing a second fluid composition based on the analysis.

13. The method of claim 1 further comprising:
changing a treatment fluid composition based on the analysis; and
introducing the treatment fluid into a wellbore penetrating a subterranean formation.

14. A method of testing the stability of deep-sea treatment fluids, the method comprising:
providing an apparatus that comprises:
a housing that comprises
a test cell being enclosed and having at least one test cell wall and one sealable opening and has a cell central axis defined along the center line of the length of the test cell,
a thermal element in thermal communication with the test cell, and
an insulation, at least a portion of the insulation being disposed about the test cell and the thermal element;
a drive connection; and
a shaft operably connected to the test cell and extending parallel to the cell central axis from the test cell through the insulation to the drive connection to which the shaft is operably connected;
providing a driving mechanism operably connected to the drive connection;
providing a sample in the test cell of the apparatus;
manipulating the test cell;
changing the sample temperature to a first temperature below room temperature;
changing the sample temperature to a second temperature above room temperature; and
analyzing the sample.

15. The method of claim 14 further comprising:
adjusting a treatment fluid composition based on the analysis; and
using the treatment fluid at a wellbore site.

16. The method of claim 14, wherein manipulating the test cell involves an action selected from the group consisting of 360° rotation about cell central axis; rocking about cell central axis; moving back and forth along cell central axis; and any combination thereof.

17. A method of testing a cement composition, the method comprising:
providing an apparatus that comprises:
a housing that comprises
a test cell being enclosed and having at least one test cell wall and one sealable opening and has a cell central axis defined along the center line of the length of the test cell,
a thermal element in thermal communication with the test cell, and
an insulation, at least a portion of the insulation being disposed about the test cell and the thermal element;
a drive connection; and
a shaft operably connected to the test cell and extending parallel to the cell central axis from the test cell through the insulation to the drive connection to which the shaft is operably connected;
providing a driving mechanism operably connected to the drive connection;
providing a cementitious sample in the test cell of the apparatus;
manipulating the test cell;
changing the sample temperature; and
monitoring the temperature within the test cell.

18. The method of claim 17, wherein the test cell comprises a coating on at least a portion of an internal cell wall.

19. The method of claim 17 further comprising:
changing a composition of solids based on the analysis.

20. The method of claim 19 further comprising:
placing the composition in a wellbore as a solid and/or as an additive in a fluid.
